(19) United States
(12) Patent Application Publication
(10) Pub. No.: US 2003/0017166 A1
(43) Pub. Date: Jan. 23, 2003

(54) Combination preparation for the therapy of immunological diseases

(76) Inventor: Juergen Lindner, Frankfurt (DE)

Correspondence Address: Finnegan, Henderson, Farabow,, Garrett & Dunner, L.L.P., 1300 I Street, N.W., Washington, DC, 20005-3315, US

(21) Appl. No.: 10/189,006

(22) Filed: Jul. 5, 2002

(30) Foreign Application Priority Data
Jul. 6, 2001 (DE) 101 32 308.5

(51) Int. Cl. A61K 39/00; A61K 31/5377; A61K 31/525

(52) U.S. Cl. 424/184.1; 514/232.8; 514/251

(57) ABSTRACT

This document describes a pharmaceutical combination preparation for treating overshooting, damaging immune reactions and degenerative processes, which preparation comprises

a) at least one antigen which is involved in an undesirable immune reaction and/or in regenerative processes;

b) at least one protein synthesis inhibitor, and, where appropriate,

c) an active compound which suppresses an acute inflammatory reaction.

The invention relates to a pharmaceutical combination preparation which can be used to treat an overshooting, damaging immune reaction, and also degenerative processes, by developing a regulatory immune response.

Overshooting, damaging immune reactions are immune reactions which are directed against endogenous and/or exogenous substances and which damage the body more than they are of use to it. Overshooting, damaging immune reactions which are directed against exogenous substances can be allergic reactions, transplant rejections or immune reactions which are directed against recombinantly modified cells or their products, such as factor VIII or insulin. Examples of overshooting, damaging reactions directed against endogenous substances are autoimmune diseases, such as multiple sclerosis, rheumatoid arthritis, pemphigus vulgaris and Hashimoto's thyroiditis. In some diseases, such as ulcerative colitis, Crohn's disease, psoriasis and arteriosclerosis, overshooting immune reactions, which damage the body more than they are of use to it, occur both against endogenous substances and against exogenous substances. Immune reactions which are directed against exogenous antigens and/or endogenous structures which have been altered by chemical compounds occur in what are termed the contact allergies, for example in the contact allergies against nickel or chromium.

Degenerative processes are understood as being diseases which are characterized by a disequilibrium between tissue synthesis and tissue breakdown. These diseases include, for example, arthrosis, dementia diseases and ulcerative colitis.

An antigen-specific effector activity of the immune system is understood as being the amplified immune reaction of the immune system against a specific antigen which is usually already known. This results in the induction of violent reactions which lead to the destruction of cells or which substantially increase an immune reaction against the recognized antigen.

An antigen-specific suppressor activity is understood as being a reaction of the immune system in which the suppressor cells react with a specific antigen, which is usually known, in such a way that the immune reaction against the recognized structure is down-regulated. If the suppressor activity outweighs the effector activity, it is then no longer possible for reactions of the immune system to be directed against the structures which are recognized by the suppressor cells.

Normally, the immune system of the body reacts to the antigen with a regulatory immune response in which an antigen-specific suppressor activity outweighs the proinflammatory or cytotoxic activity of the immune system. However, the equilibrium, which arises in this way, between the suppressor activity and the proinflammatory activity of the immune system can be crucially disturbed if an additional inflammatory reaction occurs. This then displaces the equilibrium from the antigen-specific suppressor activity toward an antigen-specific proinflammatory or cytotoxic effector activity. As soon as the inflammatory reaction has been eliminated, the equilibrium between the suppressor activity and effector activity shifts once again, as a result of the regulatory immune response, to the suppressor activity being predominant.

As a rule, diseases which are caused by overshooting, damaging immune reactions have thus far usually been treated symptomatically. In this connection, the immune reaction and its undesirable accompanying symptoms are suppressed in a relatively nonspecific manner. Immunosuppressive medicaments, such as cyclosporin A, corticoids or cyclophosphamide, suppress diseases of this nature very effectively, but they give rise to substantial side-effects. In addition, they suffer from the disadvantage that, after these medicaments have been discontinued, the disease appears once again and irreparable damage very frequently occurs during this episode of the disease. The previously existing methods for treating diseases which are caused by overshooting, damaging reactions of the immune system against particular antigens therefore either have little effect or are associated with substantial side-effects. There is therefore a great need for medicinal preparations which prevent overshooting, damaging reactions of the immune system and at the same time exhibit few side-effects. However, the treatment with such a medicinal preparation must not lead to any impairment of the immune system since, under certain pathological conditions, autoimmune reactions are of great value physiologically, for example in the case of an autoimmune reaction directed against endogenous tumor cells or virus-infected cells. However, after the tumor cells or the virus-infected cells have been destroyed, this autoimmune reaction has to come to a stop of its own accord and must not destroy any healthy endogenous tissue. Since immunosuppressive cells are known, inter alia, to produce the immunosuppressive factor TGFβ (tissue growth factor β), these cells are also able to stimulate tissue regeneration.

A pharmaceutical combination preparation has now been found, which preparation makes it possible to develop a regulatory immune response for treating an overshooting, damaging immune reaction, and also to treat degenerative processes, while avoiding an acutely inflammatory reaction, when the combination preparation comprises

a) at least one antigen which is involved in an undesirable immune reaction or in regenerative processes,

b) at least one protein biosynthesis inhibitor or nucleotide synthesis inhibitor, and, where appropriate,

c) an active compound which suppresses an acute inflammatory reaction.

Compounds which prevent the synthesis of purines or pyrimidines are termed nucleotide synthesis inhibitors. According to the invention, brequinar, mycophenolate mofetil (2-morpholinoethyl (E)-6-(1,3-dihydro-4-hydroxy-6-methoxy-7-methyl-3-oxoisobenzofuran-5-yl)-4-methyl-4-hexenoate), methotrexate, mizoribine and, in particular, a compound of the formula I or II

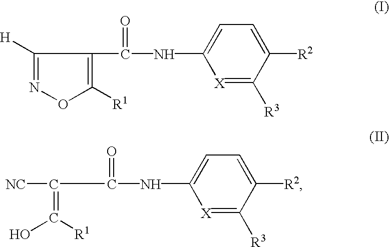

stereoisomeric forms of compounds of the formula (I) or (II) and a physiologically tolerated salt of the compound of the formula (II) in which the substituents have the following meanings:

R¹ is a) —(C₁-C₄)-alkyl,

b) —(C₃-C₅)-cycloalkyl,

c) —(C₂-C₆)-alkenyl, or

d) —(C₂-C₆)-alkynyl;

R² is a) —CF₃,

b) —O—CF₃,

c) —S—CF₃,

d) —OH,

e) —NO₂,

f) halogen,

g) benzyl,

h) phenyl,

i) —O-phenyl which is unsubstituted,

k) —CN, or

l) —O-phenyl which is monosubstituted or polysubstituted by a group selected from

R³ is a) —(C₁-C₄)-alkyl,

b) halogen, or

c) a hydrogen atom; or

X is a) a CH group,

b) a nitrogen atom,

can contains as nucleotide synthesis inhibitors.

It is also possible to use a mixture of the said nucleotide synthesis inhibitors and compounds of the formula (I) and (II) or salts of the compounds of the formula (II).

It is very particularly preferred for the combination preparation according to the invention when the N-(4-trifluoromethylphenyl)-5-methylisoxazole-4-carboxamide of the formula (I) or N-(4-trifluoromethylphenyl)-2-cyano-5-hydroxycrotonamide, 2-cyano-3-cyclopropyl-3-hydroxyacrylic acid (4-cyanophenyl)amide or N-(4-trifluoromethylphenyl)-2-cyano-3-hydroxyhept-2-en-6-ynecarboxamide, as compounds of the formula (II), are employed as the nucleotide synthesis inhibitor.

Compounds of the formulae I or II can be prepared using methods which are disclosed in the European patent applications 484 223, 529 500, 538 783 and 551 230 and the U.S. Pat. No. 4,061,767.

Leflunomide, which has previously been used as an antirheumatic, is very particularly suitable. When administered on its own, leflunomide is able to reduce the progression of rheumatoid arthritis but is not able to cure the disease. While some publications have reported that it was possible to use leflunomide to cure autoimmune diseases in experimental animals, it has turned out that the leflunomide was given prophylactically in these investigations and was consequently only able to prevent the development of an autoimmune disease and effectively suppress the symptoms of an autoimmune disease, with the leflunomide thereby increasing the survival rate of the experimental animals. The fact that the development of an autoimmune disease was prevented by the administration of leflunomide was described as being a curative therapeutic effect. However, this type of “curative” effect has nothing in common with a curative therapy of an autoimmune disease, when the disease no longer occurs after administration of the drug has been discontinued. The reports which have so far been published on the effect of leflunomide in patients suffering from rheumatoid arthritis have given no indication that an episode of the disease no longer occurs after the leflunomide had been discontinued and that the patients are cured on a long-term basis. Knowledge gained thus far with regard to the mechanism of action of leflunomide can be summarized by stating that, in vitro, leflunomide displays classical immunosuppressive effects (inhibition of pyrimidine biosynthesis, inhibition of tyrosine phosphorylation and inhibition of the signal transduction pathway for nuclear factor kappa B) In vivo, leflunomide apparently mediates its effect by way of lymphocytes. Leflunomide has only occasionally been observed to induce the phenomenon of antigen-specific tolerance; however, this only occurs sporadically and is therefore not suitable for a specific clinical application. It has been found both in clinical investigations and in investigations on experimental animals that leflunomide does not increase susceptibility to infection.

Relatively high doses of aminoglycoside antibiotics also act in a similar way to leflunomide. The effect of the aminoglycoside antibiotics such as neomycin, gentamycin or kanamycin can likewise be attributed to a shift in the equilibrium between suppressor cells and effector cells of the immune system in the direction of the suppressor cells.

Since cells of the immune system only proliferate until the underlying immune reaction has stopped, antigen-specific suppressor cells of the immune system only expand, during normal clinical use of leflunomide, until the undesirable immune response has come to a standstill. A relatively selective expansion of the immunosuppressive cells, under the influence of leflunomide, only takes place just until the directly immunosuppressive effect of leflunomide, and the suppressive effect of the newly formed suppressor cells, bring the undesirable immune reaction to a stop. If the leflunomide is then discontinued in this situation, the remaining effect of the suppressor cells is not sufficient to control the undesirable immune reaction.

Therefore, when leflunomide is used clinically in the manner which is at present customary, it is not possible to achieve any curative therapeutic result, in connection with which the leflunomide can be discontinued without the underlying immunological disease breaking out once again.

The combination preparation according to the invention now comprises, as a further essential constituent, one or more antigens which are involved in the undesirable immune reaction or in the regenerative process. The antigen(s) which the combination preparation according to the invention comprises is/are natural or artificially altered cell formations, cells, cell fragments, proteins, protein fragments or other compounds which are antigenically active. By means of simultaneously administering the antigen, against which the regulatory immune response is to be developed, and a protein biosynthesis inhibitor such as leflunomide, the proliferation of the rapidly proliferating, proinflammatory/cytotoxic effector cells of the immune system is inhibited and these cells come to a standstill in the late G1 phase. By contrast with the effector cells, leflunomide probably does not interfere with the proliferation of the more slowly proliferating suppressor cells. As a result, the activity of the suppressor cells comes, after a period of time, to outweigh that of the effector cells.

As a result of the antigen which causes/is involved in the undesirable immune reaction and/or the regenerative process being administered in addition to the leflunomide, the proliferation of antigen-specific suppressor cells of the immune system is still maintained even when the underlying undesirable immune reaction has already come to an end. It is therefore possible for the combined administration of the causative antigen and leflunomide to increase the antigen-specific suppressor cell population to such an extent that a certain excess of suppressor activity remains even after the leflunomide administration has been discontinued and the undesirable, damaging immune reaction is prevented from breaking out yet again and/or factors promoting the regeneration process continue to be released.

While the reaction situation in the immune system which the combination preparation according to the invention brings about, i.e. with the antigen-specific suppressor activity predominating, can break down briefly as the result of nonspecific inflammatory reactions, it develops once again, without any further medicinal intervention, once the nonspecific inflammatory reaction has disappeared. However, it was found that it is only possible to increase the activity of antigen-specific immunosuppressive cells when acutely inflammatory processes are avoided. For this reason, it may be necessary for the combination preparation according to the invention to comprise an active compound which suppresses an acute inflammatory reaction. In order to develop a regulatory immune response, it is necessary, therefore, to control acutely inflammatory reactions, for example by administering steroids or other substances which have an anti-inflammatory effect. These substances include antihistamines, mast cell stabilizers, such as cromoglycic acid, substances which act directly or indirectly as anti-inflammatory mediators, for example TGFβ and prostaglandin E2, or substances which suppress or antagonize the toxic mediators which are released within the context of an inflammatory process. However, the use of these anti-inflammatory active compounds must not suppress the immune reaction completely since it would not otherwise be possible to develop a regulatory immune reaction.

An antigen-specific, regulatory immune response for the curative treatment of overshooting, damaging immune reactions, and for treating degenerative processes, is consequently developed, according to the invention, by administering immunomodulating active compounds and the antigen(s) against which the undesirable immune reaction is taking place, or antigens which play a role in regenerative processes, in a manner which is restricted in time, simultaneously or in a manner which is staggered in time. It is important that no acutely inflammatory reactions take place during the development of the antigen-specific regulatory immune reaction since it is otherwise not possible to develop any regulatory immune response. It has proved to be advantageous, while continuing to administer the immunomodulated substances and regularly administer the antigen(s), to reduce the administration of anti-inflammatory active compounds stepwise, for example by slowly phasing out the steroid medication for avoiding acutely inflammatory reactions.

As soon as all the measures for avoiding acutely inflammatory reactions during the administration of the combination preparation can be discontinued, it is advisable to continue the medicinal treatment for several more weeks to permit the development of a stable regulatory immune response which prevents the reappearance of the undesirable, damaging immune response even after all the therapeutic measures have been discontinued. In the case of progressive undesirable immune reactions which have been in existence for a long time, for example rapidly progressing multiple sclerosis, it can be advantageous to make this period longer (for example 6 months) so as to ensure the formation of an adequate number of suppressor cells in the immune system, which cells still keep the effector cells under control even under slightly inflammatory conditions, for example in connection with an effect arising from influenza.

After this period of administering the combination preparation of antigen and protein biosynthesis inhibitor, it can be advantageous to continue to administer the antigens over a short period of time as a monotherapy, that is without a protein biosynthesis inhibitor, before all the therapeutic measures can be terminated.

The measures which avoid an acutely inflammatory reaction during the development of the regulatory immune response can be therapeutic principles which are coupled directly, physically or chemically, to the protein biosynthesis inhibitor, to the antigen or to a precursor of the antigen, or which are administered separately from these substances. The therapeutic principles can, for example, be substances having a steroid effect (for example fluocortolone or dexamethasone), non-steroidal anti-inflammatory agents (for example diclofenac, indometacin, ibuprofen, or inhibitors of cyclooxygenase I and/or cyclooxygenase II in general), leukotriene antagonists or inhibitors of leukotriene formation, cytokine antagonists or inhibitors of cytokine formation, mast cell stabilizers (for example cromoglycic acid), antihistamines (for example terfenadine), cyclosporin A, FK506, anti-inflammatory cytokines (for example TGFβ, IL-10, etc.), substances which induce the release of anti-inflammatory mediators (for example cyclosporin A) or anti-inflammatory fatty acids or their precursors or inhibitors of the breakdown of the anti-inflammatory fatty acids. Within the context of the combination therapy, anti-inflammatory therapeutic principles are also understood as meaning measures which suppress an acute inflammatory reaction caused by the administration of the antigen. These measures include, for example, the coating of the antigens with antibodies or fragments of antibodies, and galenic measures which avoid an acute inflammatory reaction but which permit adequate antigen presentation. The other measures for avoiding acutely inflammatory reactions which exert a negative effect on the development of a regulatory immune response also include therapeutic measures for combating and avoiding infections or nonspecific inflammatory reactions. This also includes the use of antibiotics, chemotherapeutic agents and polyvalent immunoglobulins, measures for avoiding tissue ischemia, virustatic agents and the use of therapeutic principles (including surgical principles) which combat infections generally.

The substances can be administered intravenously, by inhalation, subcutaneously, epicutaneously, rectally, intrathecally or transdermally or by way of other administration routes.

In general, it has proved to be advantageous to discontinue the protein biosynthesis inhibitor and the antigen either 2 to 3 days after all the symptoms of a self-limiting immune reaction (for example an infection such as bronchitis) have disappeared or some days after the selective induction of a self-limiting autoimmune reaction (for example induced by anti-D serum in rhesus factor-positive patients).

Infections which the immune system is unable to combat successfully have to be eliminated medicinally by means of antibiotics, chemotherapeutic agents, polyvalent immunoglobulins or immunostimulants, by means of surgical interventions or by means of physical measures. It is also necessary to use medicinal measures (for example antibiotics, chemotherapeutic agents or polyvalent immunoglobulins) or physical measures (for example exposure prophylaxis using a face-mask) to prevent a reinfection with the problem organisms since chronic inflammatory reactions lead to a breakdown in existing regulatory immune reactions and can consequently lead to the neogenesis or reappearance of undesirable harmful immune reactions.

Evidence in support of the subject-matter of the invention was provided by the following investigations: The investigations were carried out on volunteer test subjects who were suffering from a contact dermatitis to one or more metals or from an allergic reaction to grass pollen.

Contact dermatitis was selected as a model disease for the large number of T cell-mediated immune reactions (generally: overshooting damaging immune reactions) since it can be induced by applying the contact allergens to the skin and the severity of the inflammatory reaction and immune reaction can readily be assessed by observing the skin symptoms.

The effect of the combination therapy on type I allergy (allergic conjunctivitis and rhinitis) was investigated since type I allergy is an antibody-induced disease which is regulated by T cells. In the case of rhinitis and conjunctivitis, it was additionally possible for the active compounds neomycin and dexamethasone to be administered locally and topically since the surfaces are mucous membranes which enable the active compounds to be absorbed very well and the relevant components of the immune system (secondary lymphatic organs) are in direct contact with the mucous membrane. In the allergy investigations, contact with the antigen took place aerogenically, by exposure to hazelnut pollen or the pollen of grasses.

The combination preparation according to the invention can also comprise compositions or combination packages in which the constituents are contained alongside each other and are made available for the simultaneous, separate or chronologically staggered therapy of overshooting, damaging immune reactions and/or degenerative processes on one and the same human or animal body. Preference is given to administering the compound of formula I and/or II before, at the same time as, or separately in time or staggered in time in relation to administering the antigen (for example desmoglein 3) or the precursor of the antigen (for example vector which encodes the production of insulin or factor VIII) which plays a role in the overshooting, damaging immune reactions and degenerative processes (for example fragments of the joint cartilage, specific antigens of secretory tissues which degenerate in old age, such as testosterone-producing cells). For this, N-(4-trifluoromethylphenyl)-2-cyano-3-hydroxycrotonamide, for example, is firstly administered. At the same time, desmoglein 3, for example, which is an antigen which plays a central role in pemphigus vulgaris, is administered subcutaneously or intravenously. The adminstration of desmoglein 3 stimulates a proliferation of cells of the immune system which have both an intensifying and an inhibitory effect on the disease pemphigus vulgaris. The simultaneous or chronologically staggered administration of N-(4-trifluoromethylphenyl)-2-cyano-3-hydroxycrotonamide preferentially inhibits the proliferation of cells of the immune system which induce the autoimmune diseases whereas those cells which suppress the autoimmune disease continue to proliferate in a relatively undiminished manner. As a result, after a certain amount of time following the administration of desmoglein 3 and N-(4-trifluoromethylphenyl)-2-cyano-3-hydroxycrotonamide, the activity of cells of the immune system which suppress the autoimmune disease pemphigus vulgaris comes to predominate. As soon as this situation has been established, the administration of the combination preparations according to the invention can be terminated without the disease subsequently reappearing.

The preparation according to the invention can, as a dosage unit, be present in the form of medicinal forms such as inhalation systems, capsules (including microcapsules which generally do not contain any pharmaceutical excipient), tablets, including sugar-coated tablets and pills, or suppositories, with the capsule material performing the function of the excipient, and it being possible for the content to be present, for example, as a powder, a gel, a solution, an emulsion or a dispersion, when capsules are used.

However, it is particularly advantageous and simple to produce oral (peroral) formulations of the protein synthesis inhibitor/nucleotide synthesis inhibitor and of the antigen, which formulations comprise the intended quantities of the active compounds together with the desired pharmaceutical excipient. Particularly in the case of the antigen, or precursors of the antigen, it is advantageous to carry out measures, such as treatment with polyethylene glycol, which facilitate and permit absorption, in particular, and transport to the site of action, in general. A corresponding formulation (suppositories) for rectal therapy can also be employed. Transdermal/epicutaneous/buccal/scleral/nasal/pulmonary/intrathecal/ocular/inhalatory administration in the form of ointments, creams, solutions, emulsions and powders which comprise the preparation according to the invention is likewise possible. The parenteral, intravenous, intra-arterial, subcutaneous, intramuscular, intravesical, intrathecal, ocular, inhalatory or intravaginal administration of formulations which comprise the preparation according to the invention is also possible.

In addition to the active compound, ointments, pastes, creams and powders can contain the customary carrier substances, for example animal and vegetable fats, waxes, paraffins, starch, tragacanth, cellulose derivatives, polyethylene glycols, silicones, silicic acid, aluminium hydroxide, talc, zinc oxide, lactose, bentonite, calcium silicate and polyamide powder, or mixtures of these substances. The tablets, pills or granules can be prepared using methods such as pressing methods, dipping methods or fluidized bed methods or pan coating, and contain excipients and other customary auxiliary substances such as gelatin, agarose, starch (for example potato starch, corn starch or wheat starch), cellulose, such as ethyl cellulose, silicon dioxide, magnesium carbonate, various sugars, such as lactose, and/or calcium phosphates. The coating solution normally consists of sugar and/or starch syrup and usually also contains gelatin, synthetic cellulose esters, gum arabic, polyvinylpyrrolidone, pigments, surface-active substances, plasticizers and similar additives in accordance with the state of the art. Any customary flow-regulating agent, lubricant or glidant, such as magnesium stearate, and separating agent, can be used for producing the preparation forms. Preferably, the preparations have the form of shell/core tablets or multilayer tablets, with the protein synthesis inhibitor/nucleotide synthesis inhibitor being located in the shell or in the core or in a layer while the antigen, or the precursors of the antigen, is/are located in the core, in the shell or in another layer. The active compound components can also be present in delayed release form or be adsorbed onto delayed release material or be enclosed in the delayed release material (for example based on cellulose or polystyrene resin, for example hydroxyethyl cellulose). A delayed release of the active compounds can also be achieved by providing the layer or the compartment in question with customary gastric juice-insoluble coatings.

Preference is given to an administration in connection with which the antigen, or the precursors of the antigen, reach the local environment in which the immune reaction, which is to be treated, takes place. However, the antigen can also be administered systemically. The protein synthesis inhibitor/nucleotide synthesis inhibitor can be administered so that it acts either locally or systemically.

The dose to be used naturally depends on a variety of factors such as the organism to be treated (i.e. human or animal), age, weight, general state of health, the severity of the symptoms, the disease to be treated, any possible accompanying illnesses, (if present) the nature of the concomitant treatment with other drugs, or the frequency of the treatment. In general, the doses are administered several times per day, preferably once to three times per day. In this connection, the quantities of individual active compound employed follow the recommended daily dose of the given individual active compound and, in the combination preparation, should, in general, represent from 10% to 300% of the recommended daily dose, preferably from 50% to 150%, in particular 80%. A suitable treatment with the combination according to the invention consequently consists, for example, in administering one, two or three individual doses of the preparation comprising N-(4-trifluoromethylphenyl)-5-methylisoxazole-4-carboxamide or N-(4-trifluoromethyl-phenyl)-2-cyano-3-hydroxycrotonamide in a quantity of from 2 mg to 250 mg, preferably of from 5 mg to 150 mg, in particular of from 10 mg to 50 mg, particularly preferably of from 10 mg to 20 mg, and the antigen in a quantity of from 100 mg to 10 000 mg, in particular of from 1500 mg to 3000 mg.

Furthermore, the preparations according to the invention can also be employed together with other suitable active compounds, for example anti-uricopathic agents, analgesics, steroidal or nonsteroidal anti-inflammatory agents, platelet aggregation inhibitors, cytokines, cytokine agonists, cytokine antagonists or immunosuppressant compounds, such as cyclosporin A, FK 506 or rapamycin.

Pathogenesis of Contact Dermatitis:

During the sensitization phase, the foreign substances, which are usually of low molecular weight, penetrate through the skin and are taken up by dendritic Langerhans cells, either directly or after having previously been bound to proteins; they are then transported to the regional lymph nodes and presented, in these nodes, to HLA Class II-restricted T lymphocytes. Within a few days, the activation of these lymphocytes leads to an expanded contact allergen-specific T cell subpopulation, part of which is local and part of which is distributed throughout the body. Because of its T cell-dependence, contact dermatitis can be regarded as being a model disease for all overshooting, damaging immune reactions which are induced by T cells and/or result from deficient self-regulation of the immune system.

Induction of Contact Dermatitis:

The contact allergies were induced by applying preparations in white vaseline. These vaseline preparations contained either 1% cobaltII chloride or 5% nickelII sulfate.

For inducing the contact dermatitis, the vaseline preparation was applied once daily to an area of approx. 2×2 cm², usually on the upper arm or the lower arm.

In order to alleviate the itching, the test subjects were able, at their own discretion, to apply an ointment containing a local anaesthetic (e.g. EMLA ointment) to the corresponding areas of the skin.

Clinical picture of the Contact Dermatitis and Subdivision in Accordance with the Severity of the Inflammatory Reaction:

Applying the vaseline preparations induces the following skin reactions within a latency period of from several hours up to two days:

Reddening, edematous swelling of the skin, vesicles or blister formation, weeping erosions and incrustation.

The contact dermatitis was subdivided into the following levels in accordance with the extent of the inflammatory reaction:

Level 1: very minor inflammatory reaction, slight reddening of the skin;

Level 2: minor inflammatory reaction, reddening of the skin and possible itching;

Level 3: slight inflammatory reaction, reddening and edematous swelling of the skin, itching;

Level 4: moderate inflammatory reaction, reddening, edematous swelling, vesicles or blister formation, itching;

Level 5: pronounced inflammatory reaction: reddening, edematous swelling, vesicles and blister formation, weeping erosions and possible incrustation.

Using Corticoids to Control the Degree of the Inflammatory Reaction:

The severity of the inflammatory reaction which occurred was controlled by administering Ultralan® tablets systemically. When the antigen-specific regulatory immune response was induced, the inflammatory reaction was kept in the range of levels 1 and 2 by administering fluocortolone (Ultralan®). If a contact dermatitis of level 3 or higher developed despite the administration of Ultralan®, the dose of Ultralan® was then increased up to the next dosage level. If required, the dose was increased once again 2 days after the last increase in the dose. As a rule, doses of at most 10 mg/day were sufficient to restrict the symptoms of the contact allergy to levels 1 to 2.

As soon as it was no longer possible to observe any symptoms of the contact dermatitis at a given dosage level of Ultralan®, the dose was reduced on the following day and subsequently maintained once again until all the symptoms of the contact allergy had disappeared, after which the dose of Ultralan was reduced once again until, finally, it was no longer necessary to administer any Ultralan®.

As a rule, the following gradations in the daily doses of Ultralan® were employed: 20 mg, 15 mg, 10 mg, 7.5 mg, 5 mg, 2.5 mg and 0 mg.

Use of Leflunomide for Developing the Regulatory Immune Response:

An appropriate blood level of leflunomide was achieved by means of a loading dose of 100 mg on the first three consecutive days of the leflunomide therapy; after that, the leflunomide dose was reduced to 20 mg per day. Unless otherwise stated, this dose of leflunomide was maintained for up to 30 days after the time point at which the administration of flucortolone was terminated.

Subsequently, the leflunomide was eliminated at an accelerated rate from the body by means of administering 3× daily doses of 8 grams of cholestyramine over a period of 10 days.

Administering the Antigen While Developing the Regulatory Immune Response with Leflunomide in Connection with the Contact Dermatitis:

As described above, the antigen is applied to the skin once a day using a preparation prepared from white vaseline. As a rule, the antigen was administered up to the tenth day after the last administration of leflunomide.

Provocation Investigations:

In the investigations into the development of a regulatory immune response in connection with the contact dermatitis, a fresh exposure to the antigen against which a regulatory immune response had been developed was carried out no earlier than 1 month after the last contact with the antigen.

### INVESTIGATION SERIES 1

Investigation 1.1.

A contact allergy to both nickel and cobalt was first of all in each case induced in 4 test subjects who exhibited a contact allergy to these two allergens, and the dose of Ultralan® which enabled the contact allergy to be adjusted to a severity of 1 to 2 was then determined. Subsequently, the dose of Ultralan® was once again increased, for 3 days, to the precise level at which it was no longer possible to observe any skin reaction. The Ultralan® was then reduced stepwise each day until the Ultralan® had been completely discontinued. In all patients, the contact dermatitis reappeared while the Ultralan® was being reduced. As soon as a contact dermatitis of severity degree 3 developed, the exposure to the contact allergen was terminated.

Subsequently, a break of 1 month was taken during which there was no contact with the corresponding allergens and no drugs affecting the immune system were given.

After a month, all the test subjects were given a loading dose of in each case 100 mg of leflunomide on 3 consecutive days. After that, the test subjects were given a constant daily dose of 20 mg of leflunomide.

On the 4^{th} day of the leflunomide administration, a nickel-containing vaseline was applied to 2 test subjects, while the cobalt-containing vaseline was applied to the two other test subjects, for the purpose of inducing the contact dermatitis.

Despite the administration of the leflunomide, a level 3 to 4 skin reaction developed within 2 days in all the test subjects. Immediately after the skin reaction had reached level 3, the administration of steroids (Ultralan®) was begun and the contact allergy was adjusted to grade 1-2. The symptoms of the contact allergy then disappeared completely within a few days (as a rule from 3 to 5 days). The steroid dose was then reduced stepwise, as described above and while avoiding an acutely inflammatory reaction, until, finally, the steroid (Ultralan®) was no longer being administered. After the steroids had been completely discontinued, leflunomide and the antigen were administered daily over the following 30 days. During this phase, it was not possible to observe any symptoms of the contact allergy either in the test subjects who were being given the nickel-containing vaseline or in those test subjects who were being given the cobalt-containing vaseline. After 30 days, the adminstration of leflunomide was also terminated and cholestyramine was used, over 10 days, to eliminate the leflunomide at an accelerated rate. During this accelerated elimination, all the test subjects continued to be exposed to the contact allergen. Symptoms of the contact allergy were not observed in any of the test subjects during this phase.

After this period of time, the exposure to the contact allergen was also terminated.

4 weeks after this cycle of treatment had come to an end, the test subjects were once again exposed to the antigen which had been administered while the leflunomide was being administered.

Symptoms of a contact allergy did not appear in any of the test subjects over the 2 weeks during which they were provoked with the contact allergen.

Investigation 1.2.

There was then a wait of 4 weeks during which the test subjects did not have any contact with the contact allergens.

After 4 weeks, the test subjects were exposed to the antigen with which they had not had any contact during the treatment with leflunomide.

All the test subjects developed a level 3 to 4 contact dermatitis within 2 days.

Investigation 1.3.

On the 3^{rd} day, the allergen with which they had not had any contact during the treatment with leflunomide was discontinued in the case of all the test subjects; the allergen to which the test subjects had been exposed during the treatment with leflunomide was then applied instead. At the time the administration of the second contact allergen began, the affected skin areas still exhibited moderate to marked inflammatory reactions. Surprisingly, the symptoms of the contact allergy persisted as long as the contact allergen was being administered.

Investigation 1.4.

After these investigations, there was once again a break of 4 weeks. After these 4 weeks, the test subjects were exposed to the antigen to which they had been exposed during the treatment with leflunomide and against which they had not reacted approx. 4 weeks after treatment with leflunomide but against which they had reacted with a moderate to marked inflammatory reaction approx. 4 weeks previously.

On this occasion, the test subjects did not react with any contact allergy during the whole of the exposure period of 2 weeks.

Investigation 1.5.

Directly after these 2 weeks, the antigen to which the test subjects had not been exposed during the treatment with leflunomide was administered. Within 2 days, the test subjects once again developed symptoms of a level 3 to 4 contact allergy. As soon as the inflammatory reaction had developed, the antigen was discontinued and administration of the antigen which had been administered during the administration of the leflunomide was begun.

Once again, the symptoms of the contact allergy persisted over a period of 2 weeks. While the contact allergen continued to be administered after this period of 2 weeks, Ultralan® was now administered at a dose which was such that the symptoms of the contact allergy disappeared completely. As soon as the symptoms of the contact allergy had disappeared under the influence of Ultralan®, the administration of Ultralan® was reduced stepwise every day and finally discontinued completely. Even though the allergen continued to be administered to the skin for a further 2 weeks after the Ultralan® had been discontinued, no symptoms of any contact allergy appeared.

Investigation 1.6.

4 weeks later, the test subjects are exposed once again to the contact allergen which was administered during the administration of leflunomide. No symptoms of a contact allergy were observed during the first 2 weeks of the exposure to the allergen.

After these 2 weeks, the skin area was irradiated with a UV-A lamp while continuing to be exposed to the contact allergen. At the same time, a comparable skin site, to which vaseline without contact allergen was applied, was irradiated identically on the other arm of the test subjects. The UV-A irradiation caused a slight inflammatory reaction, with reddening (no edema), on the arm to which no contact allergen was applied. The symptoms of inflammation were somewhat stronger on the arm to which the contact allergen was applied even though precisely the same irradiation conditions and exposure times were selected for the two arms.

The reddening disappeared from the arm to which no contact allergen was applied after 4 days at the latest. By contrast, the inflammatory reactions on the side to which the contact allergen was applied had disappeared after 7 days at the earliest.

### INVESTIGATION SERIES 2

Investigation 2.1.

In the experiment 2, an attempt was made to develop a regulatory immune response in 2 test subjects, precisely as in experiment 1.

However, in contrast to experiment 1, a dose of Ultralan® was selected at which symptoms of the contact allergy were no longer observed. No reduction in the administration of the Ultralan® took place during the first 4 weeks. After 4 weeks, Ultralan® was completely discontinued and an attempt was made to continue to administer the leflunomide and the contact allergy. Massive symptoms of the contact allergy developed just one day after the Ultralan had been discontinued, resulting in no further investigations being carried out.

Investigation 2.2.

4 weeks later, a start was made in treating these test subjects in conformity with the combination therapy described in experiment 1. In both the test subjects, it was possible to use the treatment scheme described in experiment 1 to develop a regulatory immune response which prevented symptoms of a contact allergy appearing even in association with subsequent antigen exposure.

### INVESTIGATION SERIES 3

Investigation 3.1.

In experiment 3, the attempt to develop a regulatory immune response in conformity with the treatment scheme of experiment 1 was carried out in a further 2 test subjects suffering from contact allergy. However, in contrast to experiment 1, the treatment phase with contact allergen and leflunomide without steroids was shortened down from 30 days to 2 weeks.

4 weeks after discontinuing the leflunomide, the test subjects were exposed to the allergen. A slight reddening first of all developed 3 to 4 days after beginning the exposure to the antigen, with increasing symptoms of a contact allergy being seen over the following days.

Investigation 3.2.

A single administration of 5 mg of Ultralan®, and the simultaneous administration of leflunomide, resulted in the symptoms disappearing within one day.

Investigation 3.3.

The administration of leflunomide and the contact allergen was subsequently continued for 20 days, after which the leflunomide was eliminated in an accelerated manner, as described in experiment 1, and the administration of the contact allergen was terminated. 4 weeks after that, it was possible to demonstrate, as in experiment 1, that a stable antigen-specific regulatory immune response had developed.

### INVESTIGATION SERIES 4

Investigation 4.1.

In investigation series 4, leflunomide was used in 2 patients to develop an antigen-specific regulatory immune response selectively against one of the two different antigens (hazelnut pollen and grass pollen) against which said 2 patients had an allergic rhinitis/conjunctivitis.

First of all, the test subjects were exposed to the allergen pollens in order to verify the existence of the allergy. After that, there was a break of 1 month during which the test subjects had no contact with the corresponding allergens and no drugs affecting the immune system were administered.

After a month, the two test subjects were given a loading dose of in each case 100 mg of leflunomide on 3 consecutive days. The test subjects were then given a constant daily dose of 20 mg of leflunomide.

On the 4^{th} day of the leflunomide administration, the test subjects were exposed to one of the two allergens. The test subjects reacted immediately with allergic symptoms. The symptoms of the allergy were suppressed by administering 10 mg of Ultralan® and vasoconstrictors (Otriven® nasal drops and Yxin® eyedrops) to the extent that only slight symptoms of an allergic reaction, such as mild itching and a slightly increased flow of nasal secretion, still occurred immediately after exposure to the antigen.

Within 4 days, the symptoms of the allergy directly after exposure to the antigen disappeared completely under constant treatment with Ultralan® and leflunomide. Subsequently, the steroid dose was reduced stepwise, while maintaining the leflunomide dose and avoiding an acutely inflammatory reaction, until, finally, no steroid (Ultralan®) was any longer being administered. After the steroids had been completely discontinued, leflunomide and the antigen were administered daily for the following 30 days.

After these 30 days, the leflunomide was eliminated from the body in an accelerated manner by means of the 3× daily administration of cholestyramine over a period of 10 days. During this accelerated elimination, the daily exposure to antigen was continued.

During this procedure, no symptoms of an allergy appeared in the two test subjects.

After this treatment cycle, there was a break of one month during which the test subjects were not given any drugs for treating the allergy.

After a month, the test subjects were exposed to the allergen (pollen) to which they had been exposed during the cycle of treatment with leflunomide. Despite the continued daily exposure to allergen over 2 weeks, the test subjects did not develop any symptoms of an allergic reaction.

Investigation 4.2.

After a further 4 weeks, during which the test subjects did not have any contact with the allergens, they were exposed to the antigen (grass pollen dust 1× daily) with which they had not had any contact during the treatment with leflunomide.

Both the test subjects developed symptoms of an allergy within 2 days.

Investigation 4.3.

As soon as the test subjects exhibited symptoms of an allergy, together with the symptoms of an inflammatory reaction, they were exposed, for a further 2 days, both to the allergen which was administered in combination with the leflunomide and to the allergen which was not administered in combination with the leflunomide. On the 3^{rd} and 4^{th} days, only that antigen was administered which had originally been administered in combination with the leflunomide. Symptoms of an allergy developed both on the 3^{rd} day and on the 4^{th} day. On the following 2 days, dexamethasone nasal spray and eyedrops were administered prior to exposure to the antigen, thereby ensuring that it was not possible for any inflammatory reactions to develop. These measures also served to consume the antibodies which can be located, over a relatively long period of time, on the surfaces of cells of the immune system.

Investigation 4.4.

After a further 4 weeks, the test subjects were exposed once again to the antigen to which they had been exposed during the leflunomide administration. No symptoms of an allergy developed even after 2 weeks of daily exposure to the antigen.

### INVESTIGATION SERIES 5

Investigation 5.1.

In investigation series 5, as in investigation series 4, an antigen-specific regulatory immune response was developed in 2 patients against one of the two different antigens (hazelnut pollen and grass pollen) against which said two patients had an allergic rhinitis/conjunctivitis.

During the allergen exposure, dexamethasone-containing nasal sprays and eyedrops were used to reduce the immune reaction to the extent that no acute inflammatory symptoms were any longer observed. In addition, the test subjects were able to use vasoconstrictors, such as Otriven® nasal drops and Yxin® eyedrops, to alleviate acute symptoms. However, it was possible for a slightly increased formation of nasal secretion and slight itching of the eyes to occur during the allergen exposure.

Neomycin sulfate was used, at a concentration of 50 mg/ml in physiological sodium chloride solution, as an immunomodulatory substance, as eyedrops and as a metered nasal spray. During the allergen exposure (season of airborne pollen), the neomycin solution was initially administered every 3 hours during the day. As soon as symptoms of an allergic reaction, such as severe itching, tears, flow of nasal secretion or sneezing impulses, occurred, this inflammatory reaction was suppressed, within the first 5 days, by using the dexametasone eyedrops, the dexamethasone nasal spray, xylometazoline nasal drops and Yxin® eyedrops. The steroids and the vasoconstrictors were administered as required.

Within the first 5 days, the requirement for dexamethasone adminstrations decreased from an average of 4 administrations per day down to 2 administrations per day, given in the morning and in the afternoon.

Over the following 4 days, cromoglycic acid was administered instead of dexamethasone; at the same time, it was possible to reduce the administration of the neomycin solution to in the morning, at midday and in the evening. After these 4 days, the cromoglycic acid was no longer required. The administration of the neomycin solution was continued unchanged for a further 2 weeks. After a week, the test subjects were exposed twice daily to the antigen in an intensified manner (direct exposure to hazelnut pollen). When this was done, no allergy symptoms appeared in either of the test subjects.

After this treatment cycle, there was a break of a month during which the test subjects were not given any drugs for treating the allergy.

After a month, the test subjects were exposed to the allergen (pollen) to which they had been exposed during the cycle of treatment with neomycin. The test subjects did not exhibit any allergy symptoms during this daily exposure to allergen, which lasted 2 weeks.

Investigation 5.2.

4 weeks later, during which time the test subjects had not had any contact with the allergens, they were then exposed to the antigen (dust of the grass pollens, 1× daily) with which they had not had any contact during the treatment with neomycin.

Both the test subjects developed symptoms of an allergy within 2 days.

Investigation 5.3.

As soon as the test subjects exhibited symptoms of an allergy together with the symptoms of an inflammatory reaction, they were exposed for a further 2 days both to the allergen which had been given in combination with neomycin and to the antigen which had not been given in combination with neomycin. On the 3^{rd} day and on the 4^{th} day, only that antigen which had originally been given in combination with neomycin was administered. Symptoms of an antigy developed both on the 3^{rd} day and on the 4^{th} day. In the following 2 days, dexamethasone was administered prior to exposure to the antigen, thereby ensuring that it was not possible for any inflammatory reactions to develop. These measures also had the function of consuming the antibodies which are present, over a relatively long period of time, on the surface of cells of the immune system.

Investigation 5.4.

After another break of 4 weeks, the test subjects were exposed once again to the antigen to which they had been exposed during the neomycin administration. Even after 2 weeks of daily exposure to the antigen, they had not developed any allergy symptoms.

Conclusions from the Investigations:

Investigation series 1.1. shows that a combination treatment consisting of leflunomide and the inducing allergen can elicit a lasting nonreactivity towards the administered antigen. This nonreactivity was developed in connection with an immune reaction which was already active. It was likewise demonstrated that the nonreactivity of the immune system cannot be achieved simply by reducing the administration of Ultralan® stepwise. Investigation 1.2. provides evidence that this nonreactivity only exists toward the antigen which was administered during the treatment with leflunomide and that the reactivity of the immune system was not damaged nonspecifically. The 1.2. investigations prove that the combination treatment elicited an antigen-specific nonreactivity.

Investigation 1.3. provides evidence that the antigen-specific nonreactivity is lost under inflammatory conditions. It can be concluded from this that the combination treatment of leflunomide and an antigen does not result in the immune system losing any possibilities of reacting; on the contrary, these possibilities are once again available when inflammatory reactions occur. Investigation 1.3. therefore also provides evidence that a combination treatment of the antigen and the leflunomide also develops an antigen-specific regulatory immune response. Investigation 1.4. provides evidence that the antigen-specific nonreactivity of the immune system reappears, without any further therapeutic measures, after the inflammatory reaction has regressed. Investigation 1.5. provides evidence that the antigen-specific nonreactivity also reappears when drugs are used to briefly suppress the inflammatory reaction.

Investigation 1.6. proves that not only immunologically induced inflammatory reactions, but also nonspecific inflammatory reactions quite generally, can cause the antigen-specific nonreactivity to break down. In addition, this investigation proves that, in the case of moderate inflammatory reactions, the nonreactivity develops once again of its own accord, despite contact with the antigen, after the cause of the inflammatory reaction has been removed.

Investigation series 2.1. provides evidence that the immune reaction ought not to be completely suppressed during the development of the antigen-specific regulatory immune response. Investigation 2.2. provides evidence that the test subjects treated in 2.1. are not cases who are resistant to therapy.

Investigation series 3 provides evidence that, even after the steroid treatment has been completely discontinued, the antigen-specific regulatory immune response only redevelops, in dependence on time, when leflunomide and the antigen are administered at the same time. These investigations also provide evidence that administering leflunomide on its own during an immune reaction does not develop any antigen-specific regulatory immune response which is sufficiently stable. They provide evidence that, even after all the symptoms of the disease have disappeared, it is still necessary to continue to administer antigen in combination with leflunomide so as to ensure the establishment of an antigen-specific regulatory immune reaction which is sufficiently stable and which prevents the appearance of the undesirable overshooting, harmful immune reactions even after the leflunomide has been discontinued.

Investigations series 4 demonstrated that it is possible to use the combination treatment of leflunomide and an antigen to develop an antigen-specific regulatory immune response even in the case of antibody-mediated immune reactions.

Investigation series 5 provided evidence that an antigen-specific regulatory immune response can be developed using neomycin eyedrops and a neomycin nasal spray over a limited period of time, while avoiding acute inflammatory reactions and maintaining contact with the antigen in question. As in the case of the regulatory immune response which was developed using leflunomide, the regulatory immune response which was developed using neomycin also demonstrates that, while nonspecific inflammatory reactions can cause the nonreactivity of the immune system to break down, this nonreactivity develops once again, of its own accord, after the cause of the nonspecific inflammation has disappeared.

1. A pharmaceutical combination preparation, which comprises
a) at least one antigen which is involved in an undesirable immune reaction or in regenerative processes, or a precursor of this antigen,
b) at least one protein biosynthesis inhibitor and/or at least one nucleotide synthesis inhibitor, and, where appropriate,
c) an active compound which suppresses an acute inflammatory reaction.

2. The combination preparation as claimed in claim 1, which comprises, as the antigen, natural or artificially altered cell formations, cells, cell fragments, proteins, protein fragments, precursors of antigens or other compounds which are active antigenically.

3. The combination preparation as claimed in claims 1 and 2, which comprises, as nucleotide synthesis inhibitors, brequinar, mycophenolate mofetil (2-morpholinoethyl (E)-6-(1,3-dihydro-4-hydroxy-6-methoxy-7-methyl-3-oxoisobenzofuran-5-yl)-4-methyl-4-hexanoate), methotrexate, mizoribine and, in particular, a compound of the formula I or II 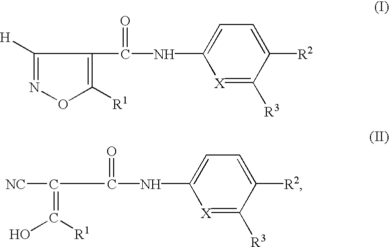
stereoisomeric forms of compounds of formula (I) or (II) and a physiologically tolerated salt of the compound of the formula (II), in which the substituents have the following meanings:
R¹ is a) —(Cₗ-C₄)-alkyl,
b) —(C₃-C₅)-cycloalkyl,
c) —(C₂-C₆)-alkenyl, or
d) —(C₂-C₆)-alkynyl;
R² is a) —CF₃,
b) —O—CF₃,
c) —S—CF₃,
d) —OH,
e) —NO₂,
f) halogen,
g) benzyl,
h) phenyl,
i) —O-phenyl which is unsubstituted,
j) —CN, or
k) —O-phenyl which is monosubstituted or polysubstituted by a group selected from
1) (C₁-C₄)-alkyl,
2) halogen,
3) —O—CF₃ and
4) —O—CH₃;
R³ is a) —(C₁-C₄)-alkyl,
b) halogen, or
c) a hydrogen atom; or
X is a) a CH group,
b) a nitrogen atom,
or a mixture of said nucleotide synthesis inhibitors and compounds of the formula (I) and (II) or of salts of the compounds of the formula (II).

4. The combination preparation as claimed in claims 1 to 3, which comprises, as nucleotide synthesis inhibitor, the N-(4-trifluoromethylphenyl)-5-methyl-isoxazole-4-carboxamide of the formula (I) or N-(4-trifluoromethylphenyl)-2-cyano-5-hydroxycrotonamide, 2-cyano-3-cyclopropyl-3-hydroxyacrylic acid (4-cyanophenyl)amide or N-(4-trifluoromethylphenyl)-2-cyano-3-hydroxyhept-2-en-6-ynecarboxamide, as compounds of the formula (II).

5. The combination preparation as claimed in claims 1 to 4, which comprises, as protein biosynthesis inhibitor, leflunomide, an aminoglycoside antibiotic or a derivative of the previously mentioned active compounds.

6. The combination preparation as claimed in claims 1 to 5, which comprises, as an active compound which suppresses an acute inflammatory reaction, a steroid, a nonsteroidal anti-inflammatory agent, a leukotriene antagonist, a cytokine antagonist, a mast cell stabilizer, an antihistamine, a cyclosporin, FK 506, an anti-inflammatory cytokine or an anti-inflammatory fatty acid, or their precursors or inhibitors.

7. The combination preparation as claimed in claim 6, which comprises an active compound which is suitable for suppressing an inflammatory reaction which is associated with an infection.

8. The combination preparation as claimed in claims 1 to 7, wherein, in this preparation, the individual active compounds are provided for the simultaneous, separate or chronologically staggered treatment of overshooting, damaging immune reactions and/or degenerative processes.

9. The combination preparation as claimed in claims 1 to 8, which comprises at least one further active compound, a galenic auxiliary substance and/or a carrier substance.

10. The use of the combination preparation as claimed in claims 1 to 9, wherein the preparation is used for treating overshooting, damaging immune reactions and/or degenerative processes.
